(19) United States
(12) Patent Application Publication
(10) Pub. No.: US 2003/0166544 A1
(43) Pub. Date: Sep. 4, 2003

(54) Use of ADNP for the treatment of glaucomatous optic neuropathy

(76) Inventors: Abbot F. Clark, Arlington, TX (US); Debra L. Shade, Benbrook, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/164,432

(22) Filed: Jun. 6, 2002

Related U.S. Application Data
(63) Continuation-in-part of application No. 09/921,029, filed on Aug. 2, 2001.
(60) Provisional application No. 60/230,964, filed on Sep. 7, 2000.

(51) Int. Cl. A61K 38/17; A61K 38/10; A61K 38/08

(52) U.S. Cl. 514/12; 514/13; 514/14; 514/15; 514/16

(57) ABSTRACT

Methods for preventing and treating glaucomatous optic neuropathy using peptides derived from or related to Activity Dependent Neuroprotective Factor (ADNP) are disclosed.

This application is a continuation-in-part of U.S. Ser. No. 09/921,029, filed Aug. 2, 2001, which claims priority from U.S. Provisional Application Serial No. 60/230,964 filed Sep. 7, 2000.

The present application is directed to the use of Activity Dependent Neuroprotective Protein (ADNP) for the treatment of glaucomatous optic neuropathy.

### BACKGROUND OF THE INVENTION

The glaucomas are a heterogeneous group of optic neuropathies characterized by the cupping of the optic nerve head, thinning of the retinal nerve fiber layer due to loss of retinal ganglion cells, and specific pathogenetic changes in the visual field. Although elevated intraocular pressure (IOP) is an important risk factor for the development of many common forms of glaucoma (Sommer et al. 1991), the phenomenon of normal tension glaucoma has been clinically established in ophthalmology (Flammer 1990). Normal tension glaucoma is characterized by an intraocular pressure which is in the normal range, i.e., not increased, but in which the optic nerve disk is pathologically excavated and the field of vision is impaired.

At the present time glaucoma, including normal tension glaucoma, is treated by medically and/or surgically lowering elevated pressure; however, even when IOP is maintained with in a normal range visual field loss may progress. Degeneration involving retinal ganglion cells may be related to compression of the nerve fiber bundles, excitotoxicity, ischemia, or other as yet unrecognized causative factors. Thus, factors other than IOP may play a role in determining both the occurrence and rate of progression of retinal ganglion cell death and subsequent visual field loss.

Using laboratory models, including ischemia, optic nerve crush, optic nerve transection, and cultured retinal ganglion cells (Adachi et al. 1998; Yoles et al. 1998; Di Polo et al. 1998; Caprioli et al. 1996; Woldemussie et al. 1997), various pharmacological agents have been tested as potential neuroprotective approaches designed to reduce retinal ganglion cell loss. These approaches have suggested that antagonism of excitotoxicity or supplementation of neurotrophic factors can protect retinal ganglion cells from degeneration in animal models. The use of compounds capable of reducing glutamate toxicity (WO 94/13275) and polyamine antagonists (U.S. Pat. No. 5,710,165) to protect retinal ganglion cells and reduce visual field loss associated with glaucoma have been disclosed. The protective effect of MK-801, a glutamate antagonist, in a rat model of ocular hypertension, was reported. (Chaudhary et al. 1998).

Activity dependent neurotrophic factor (ADNF) is a glia-derived protein which has been found to be neuroprotective at femtomolar concentrations. ADNF is both a regulator of activity dependent neuronal survival and a neuroprotectant (Gozes et al. 1997; Brenneman et al. 1998; and WO 96/11948). Gozes, et al., also describe ADNF as protective against a broad range of toxins relative to Alzheimer's disease, human immunodeficiency virus (HIV), excitotoxicity, and electrical blockade. They propose the compound for development against neurodegeneration (Gozes et al. 1996). U.S. Pat. No. 5,767,240 discloses that ADNF protein increases survival of activity dependent spinal cord nerves and cerebral cortical nerves, and prevents neuronal cell death resulting from HIV. A recent publication by Guo et al. (1999), discloses that certain neurotrophic factors, including ADNF, protect hippocampal neurons which contain presenilin-1 mutations from glutamate induced cytotoxicity. WO 98/35042 discloses the use of ADNF III for conditions leading to neuronal cell death. ADNF III is a separate gene from ADNF having a predicted molecular weight of about 123 kDa and having neurotrophic/neuroprotective activity (WO 01/09311; Gozes et al. 2000). ADNF III has also been referred to as activity dependent neuroprotective factor (ADNP). None of these references disclose or suggest the use of ADNF and related compounds for use in treating glaucoma.

ADNP is a glial mediator of vasoactive intestinal peptide (VIP)-associated neuroprotection (Gozes et al. 2000). It has been disclosed for the treatment of neurological deficiencies and for the prevention of cell death associated with: gp 120, the envelope protein from the human immunodeficiency virus (HIV), N-methyl-D-aspartic acid, tetrodotoxin, and β-amyloid peptide (WO 98/35042). Pathologies said to benefit from the therapeutic applications set forth in WO 98/35042 are set forth on page 60 of the publication. Pathologies include retinal neuronal degeneration, but there is no characterization of which retinal disease this compound might target. Glaucomatous optic neuropathy is not disclosed.

### SUMMARY OF THE INVENTION

The present invention overcomes these and other drawbacks of the prior art by providing, in one aspect, a method for treating glaucomatous optic neuropathy by administering to a patient in need thereof a composition containing a pharmaceutically effective amount of a peptide, polypeptide or protein including a sequence consisting of at least 8 contiguous amino acids from SEQ ID NO:2. Preferably, the peptide or polypeptide includes the sequence as set forth in SEQ ID NO:4 or SEQ ID NO:8.

It will be understood that the phrase “at least 8 contiguous amino acids” includes all lengths of peptide or polypeptide that are 8 amino acids in length or longer, up to the full length of the protein (SEQ ID NO:2). That is, it includes peptides that are 9, 10, 11, 12, etc., 20, 21, 22, 23, etc., 40, 41, 42, 43, etc. 100, 101, 102, etc., 150, 151, 152 amino acids etc. and so on up to the full length of the protein (1102 amino acids in length).

In another aspect, the present invention provides a method for treating glaucomatous optic neuropathy by administering to a patient in need thereof a composition containing a pharmaceutically effective amount of a peptide, polypeptide or protein including a sequence consisting of a sequence as set forth in SEQ ID NO:3, SEQ ID NO:5, SEQ ID NO:6, or SEQ ID NO:7.

### BRIEF DESCRIPTION OF THE DRAWINGS

The following drawing forms part of the present specification and is included to further demonstrate certain aspects of the present invention. The invention may be better understood by reference to this drawing in combination with the detailed description of specific embodiments presented herein.

FIG. 1 illustrates a comparison of the amino acid sequence of ADNP and ADNF III. As shown, the amino acid sequence of ADNP contains a 33 amino acid insert that is not present in the sequence of ADNF III.

### DETAILED DESCRIPTION OF PREFERRED EMBODIMENTS

Glaucoma is a heterogeneous group of optic neuropathies that share certain clinical features. The loss of vision in glaucoma is due to the selective death of retinal ganglion cells in the neural retina that is clinically diagnosed by characteristic changes in the visual field, nerve fiber layer defects, and a progressive cupping of the ONH. One of the main risk factors for the development of glaucoma is the presence of ocular hypertension (elevated intraocular pressure, IOP). IOP also appears to be involved in the pathogenesis of normal tension glaucoma where patients have what is often considered to be normal IOP. The elevated IOP associated with glaucoma is due to elevated aqueous humor outflow resistance in the trabecular meshwork (TM), a small specialized tissue located in the iris-corneal angle of the ocular anterior chamber. Glaucomatous changes to the TM include a loss in TM cells and the deposition and accumulation of extracellular debris including plaque-like material. In addition, there also are changes that occur in the glaucomatous optic nerve head. In glaucomatous eyes, there are morphological and mobility changes in ONH glial cells. In response to elevated IOP and/or transient ischemic insults, there is a change in the composition of the ONH extracellular matrix and alterations in the glial cell and retinal ganglion cell axon morphologies.

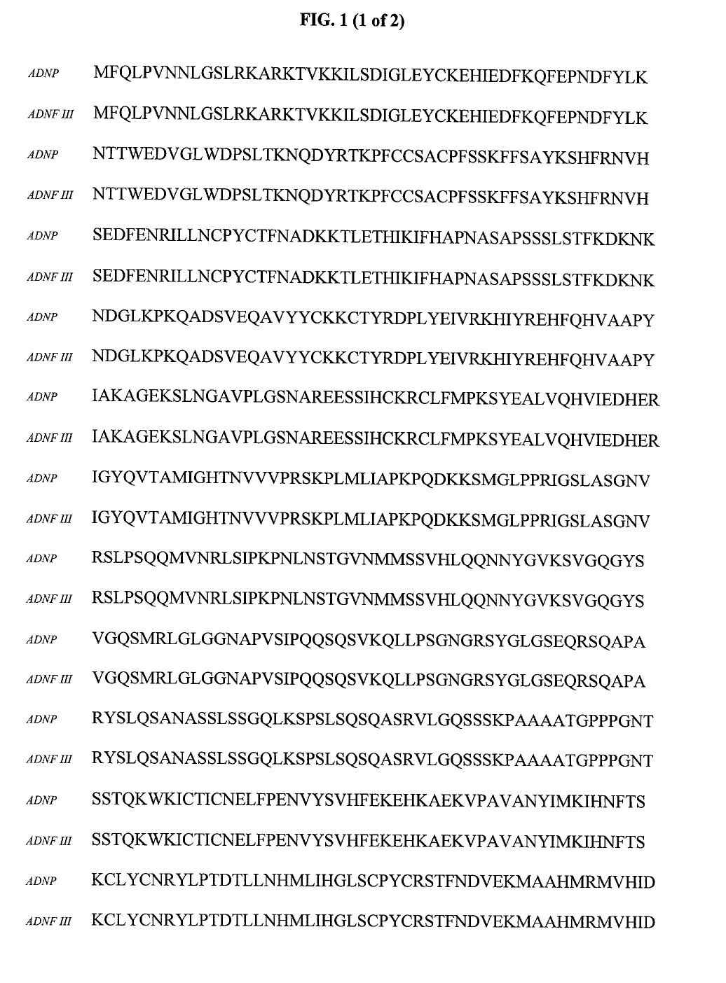

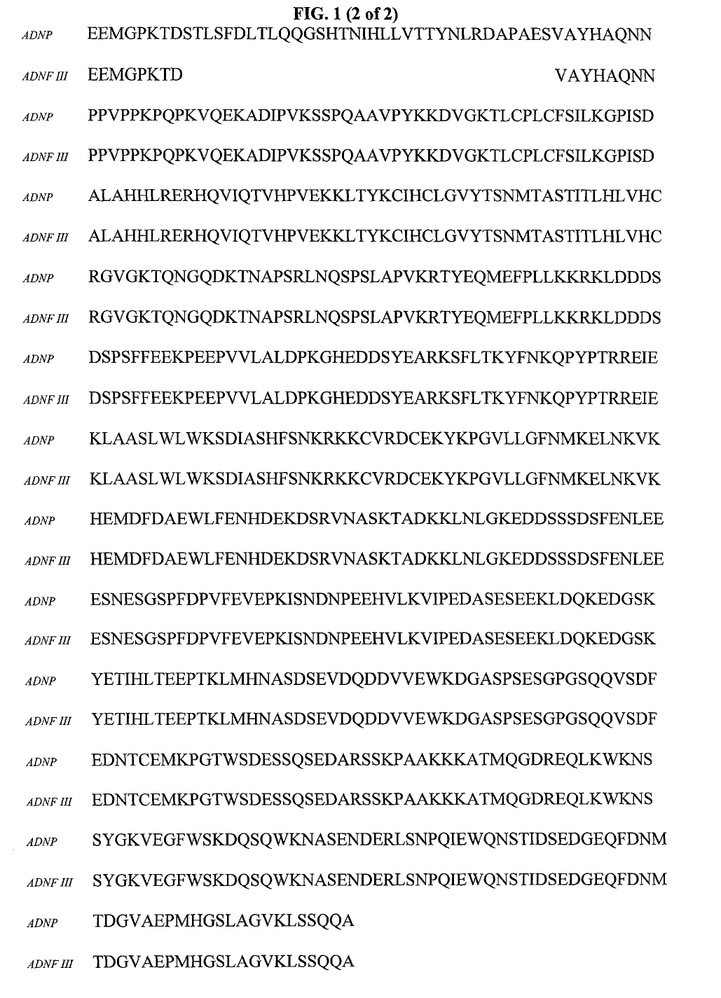

As with previously described ADNF I, ADNF III exhibits potent neuroprotective effects, with the EC₅₀ of such neuroprotective effects being in the femtomolar range. Based on the recognized homology between ADNF I and hsp60, a heat shock protein, and PIF1, a DNA repair protein, these two epitopes were utilized to prepare antibodies which, in turn, were used to screen a mouse cDNA expression library to identify the new neuroprotective polypeptide ADNP. Human ADNP has also been cloned. The human nucleotide sequence of ADNP is set forth in SEQ ID NO:1 and the amino acid sequence is set forth in SEQ ID NO:2. It has been found that the amino acid sequence of ADNP contains a 33 amino acid insert (SEQ ID NO:9) as compared to the amino acid sequence of ADNF III. FIG. 1 provides a comparison of the sequences of ADNF III and ADNP, illustrating the insert.

The present inventors have discovered that administering an ADNP peptide or protein consisting of at least eight contiguous amino acids from SEQ ID NO:2 provides a means for protection of both the retina and the optic nerve/nerve head of glaucoma patients. Thus, ADNP is believed to be useful to treat glaucomatous optic neuropathy. As used herein ADNP means ADNP, ADNP peptides, ADNP peptidomimetics, ADNP small molecule analogues, and any agent that upregulates endogenous ADNP, or an expression vector which induces ADNP expression.

Based on the homology between ADNF I and hsp60 to ANDP, a polypeptide consisting of eight contiguous amino acids of SEQ ID NO:2 was synthesized that exhibited structural homology to hsp60 and to the previously described ADNF-derived active peptide SALLRSIPA (SEQ ID NO:3). This ADNP polypeptide is 8 amino acids in length and has the sequence NAPVSIPQ, i.e., Asn-Ala-Pro-Val-Ser-Ile-Pro-Gln (SEQ ID NO:4). Other sequences contemplated to be useful in the practice of the present invention include sequences comprising any one of the following sequences: GSALLRSIPA (SEQ ID NO:5), VLGGGSALLRSIPA (SEQ ID NO:6), VEEGIVLGGGSALLRSIPA (SEQ ID NO:7), TALLRTIPA (SEQ ID NO:8) and a sequence essentially as set forth in SEQ ID NO:2.

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2. The term “biologically functional equivalent” is well understood in the art. Accordingly, sequences that have between about 70% and about 80%; or more preferably, between about 81% and about 90%; or even more preferably, between about 91% and about 99%; of amino acids that are identical or functionally equivalent to the amino acids of SEQ ID NO:2 will be sequences that are “essentially as set forth in SEQ ID NO: 1.”

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of neuroprotective activity.

Thus, in one aspect, the present invention provides a method for treating glaucomatous optic neuropathy by administering a composition including a peptide or polypeptide consisting of at least 8 contiguous amino acids from SEQ ID NO:2. It is further contemplated that the composition could include a peptide comprising an amino acid sequence as set forth in SEQ ID NO:4, SEQ ID NO:8 or SEQ ID NO:9. It is also contemplated that the composition could include the full sequence of ADNP, as set forth in SEQ ID NO:2. In additional embodiments, the present invention provides a method for treating glaucomatous optic neuropathy by administering a composition including a peptide or polypeptide consisting of SEQ ID NO: 3, SEQ ID NO:5, SEQ ID NO:6, or SEQ ID NO:7.

The agents of this invention, can be incorporated into various types of ophthalmic formulations for delivery to the eye (e.g., topically, intracamerally, or via an implant). The agents are preferably incorporated into topical ophthalmic formulations for delivery to the eye. The agents may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving an agent in a physiologically acceptable isotonic aqueous buffer. Further, the ophthalmic solution may include an ophthalmologically acceptable surfactant to assist in dissolving the agent. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the agent in a hydrophilic base prepared from the combination of, for example, carbopol-974, or the like, according to the published formulations for analogous ophthalmic preparations; preservatives and tonicity agents can be incorporated.

The agents are preferably formulated as topical ophthalmic suspensions or solutions, with a pH of about 4 to 8. The establishment of a specific dosage regimen for each individual is left to the discretion of the clinicians. The agents will normally be contained in these formulations in an amount 0.01% to 5% by weight, but preferably in an amount of 0.05% to 2% and most preferably in an amount 0.1 to 1.0% by weight. The dosage form may be a solution, suspension microemulsion. Thus, for topical presentation 1 to 2 drops of these formulations would be delivered to the surface of the eye 1 to 4 times per day according to the discretion of a skilled clinician.

The agents can also be used in combination with other agents for treating glaucoma, such as, but not limited to, β-blockers, prostaglandin analogs, carbonic anhydrase inhibitors, α₂ agonists, miotics, and neuroprotectants.

The agents can be administered in a variety of ways to achieve therapeutic concentrations at the retina and/or optic nerve head. For example, the agent may be delivered directly to the eye (for example: topical ocular drops or ointments; slow release devices in the cul-de-sac or implanted adjacent to the sclera or within the eye; periocular, conjunctival, sub-Tenons, intracameral or intravitreal injections) or parenterally (for example: orally; intravenous, subcutaneous or intramuscular injections; dermal delivery; etc.) using techniques well known by those skilled in the art. The following are examples of possible formulations embodied by this invention.

| | | |
|---|---|---|
| (a) | Topical ocular formulation | wt. % |

| | |
|---|---|
| ADNP peptide | 0.005-5.0 |
| Tyloxapol | 0.01-0.05 |
| HPMC | 0.5 |
| Benalkonium chloride | 0.01 |
| Sodium chloride | 0.8 |
| Edetate disodium | 0.01 |
| NaOH/HCl | q.s. pH 7.4 |
| Purified water | q.s. 100 mL |

It is further contemplated that the compounds of the invention could be formulated in intraocular insert devices.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

United States Patents

U.S. Pat. No. 5,710,165

U.S. Pat. No. 5,767,240

Foreign Patents and Published Patent Applications

WO 94/13275

WO 96/11948

WO 98/35042

WO 01/09311

Other Publications

Adachi, K. et al., Eur. J Pharmacol., 350:53-57 (1998).

Brenneman et al., JPET, 285:619-627 (1998).

Caprioli, J. et al., Invest. Ophthalmol. Vis. Sci., 37:2376-2381 (1996).

Chaudhary, P. et al., Brain Res. 792:154-158 (1998).

Di Polo, A. et al., Proc. Natl. Acad. Sci, USA 95:3978-83 (1998).

Flammer, J., Fortschr. Ophthalmol. 87:187 (1990).

Gozes et al., Developmental Brain Research, 99(2):167-175 (1997).

Gozes et al., J. Molecular Neuroscience 7(4):235-244 (1996).

Gozes and Brenneman, J. Molecular Neuroscience 14:61-68 (2000).

Guo et al., Proc. Natl. Acad. Sci, 96:4125-4130 (1999).

Park et al., Biochem. Biophys. Res. Commun. 284(4):966-971 (2001).

Sommer, A. et al., Arch. Ophthalmol. 109:1090-1095 (1991).

Woldemussie, E. et al., Invest. Ophthalmol. Vis. Sci., 38:S100 (1997).

Yoles, E. et al., Arch. Opthalmol., 116:906-910 (1998).

We claim:

1. A method for treating glaucomatous optic neuropathy, said method comprising administering to a patient in need thereof a composition comprising a pharmaceutically effective amount of a peptide or protein comprising a sequence consisting of at least 8 contiguous amino acids from SEQ ID NO:2.

2. The method of claim 1, wherein the peptide comprises SEQ ID NO:4.

3. The method of claim 1, wherein the composition comprises a peptide comprising a sequence consisting of at least 9 contiguous amino acids from SEQ ID NO:2.

4. The method of claim 3, wherein the peptide comprises SEQ ID NO:8.

5. A method for treating glaucomatous optic neuropathy, said method comprising administering to a patient in need thereof a composition comprising a pharmaceutically effective amount of a peptide or protein comprising a sequence consisting of a sequence selected from the group consisting of SEQ ID NO:3, SEQ ID NO:5, SEQ ID NO:6, and SEQ ID NO:7.


SEQUENCE LISTING (9 sequences)

SEQ ID NO: 1 - RNA, 4713 nt, homo sapiens
cggccgcggcgcgagccggaguccgccgagccggagcgcgacgaggccccgggcgcgcccuccccgcugccgccaccgccgugccgccgccauccgcccgccgccgccgccgcuguccggcccccgagcacgccggccccgcgcgcgccucgaggccgagucaaggugugagaugcacaaugcgaaaccuaggccccagcuuuuacaccaugaugcgcaggguuguacuuuuuguacugaacugauagguggccuagugguuaugcccuguacuaccauuuugaggaucuggacuccguuuccugccuugcucuuuggaccacauugucaauucacaccgaaacuauguuccaacuuccugucaacaaucuuggcaguuuaagaaaagcccggaaaacugugaaaaaaauacuuagugacauuggguuggaauacuguaaagaacauauagaagauuuuaaacaauuugaaccuaaugacuuuuauuugaaaaacacuacaugggaggauguaggacugugggacccaucacuuacgaaaaaccaggacuaucggacaaaaccuuucugcugcagcgcuuguccauuuuccucaaaauucuucucugccuacaaaagucauuuccgcaauguccauagugaagacuuugaaaauaggauucuccuuaauugccccuacuguaccuucaaugcagacaaaaagacuuuggaaacacacauuaaaauauuucaugcuccgaacgccagcgcaccaaguagcagccucagcacuuucaaagauaaaaacaaaaaugauggccuuaaaccuaagcaggcugacaguguagagcaagcuguuuauuacuguaagaagugcacuuaccgagauccucuuuaugaaauaguuaggaagcacauuuacagggaacauuuucagcauguggcagcaccuuacauagcaaaggcaggagaaaaaucacucaauggggcaguccccuuaggcucgaaugcccgagaagagaguaguauucacugcaagcgaugccuuuucaugccaaaguccuaugaagcuuugguacagcaugucaucgaagaccaugaacguauaggcuaucaggucacugccaugauugggcacacaaauguagugguuccccgauccaaacccuugaugcuaauugcucccaaaccucaagacaagaagagcaugggacucccaccaaggaucgguucccuugcuucuggaaauguccggucuuuaccaucacagcagauggugaaucgacucucaauaccaaagccuaacuuaaauucuacaggagucaacaugauguccaguguucaucugcagcagaacaacuauggagucaaaucuguaggccaggguuacaguguuggucagucaaugagacugggucuagguggcaacgcaccaguuuccauuccucaacaaucucagucuguaaagcaguuacuuccaaguggaaacggaaggucuuaugggcuugggucagagcagaggucccaggcaccagcaagauacucccugcagucugcuaaugccucuucucucucaucgggccaguuaaagucuccuucccucucucagucacaggcauccagaguguuaggucaguccaguuccaaaccugcugcagcugccacaggcccucccccagguaacacuuccucaacucaaaaguggaaaauauguacaaucuguaaugagcuuuuuccugaaaaugucuauagugugcacuucgaaaaagaacauaaagcugagaaagucccagcaguagccaacuacauuaugaaaauacacaauuuuacuagcaaaugccucuacuguaaucgcuauuuacccacagauacucugcucaaccauauguuaauucauggucugucuuguccauauugccguucaacuuucaaugauguggaaaagauggccgcacacaugcggaugguucacauugaugaagagaugggaccuaaaacagauucuacuuugaguuuugauuugacauugcagcaggguagucacacuaacauccaucuccugguaacuacauacaaucugagggaugccccagcugaaucuguugcuuaccaugcccaaaauaauccuccaguuccuccaaagccacagccaaagguucaggaaaaggcagauaucccuguaaaaaguucaccucaagcugcagugcccuauaaaaaagauguugggaaaacccuuuguccucuuugcuuuucaauccuaaaaggacccauaucugaugcacuugcacaucacuuacgagagaggcaccaaguuauucagacgguucauccaguugagaaaaagcucaccuacaaauguauccauugccuugguguguauaccagcaacaugaccgccucaacuaucacucugcaucuaguucacugcaggggcguuggaaagacccaaaauggccaggauaagacaaaugcacccucucggcuuaaucagucuccaagucuggcaccugugaagcgcacuuacgagcaaauggaauuucccuuacugaaaaaacgaaaguuagaugaugauagugauucacccagcuucuuugaagagaagccugaagagccuguuguuuuagcuuuagaccccaagggucaugaagaugauuccuaugaagccaggaaaagcuuucuaacaaaguauuucaacaaacagcccuaucccaccaggagagaaauugagaagcuagcagccaguuuaugguuauggaagagugacaucgcuucccauuuuaguaacaaaaggaagaaguguguccgugauugugaaaaguacaagccuggcguguugcugggguuuaacaugaaagaauuaaauaaagucaagcaugagauggauuuugaugcugaguggcuauuugaaaaucaugaugagaaggauuccagagucaaugcuaguaagacugcugacaaaaagcucaaccuugggaaggaagaugacaguuccucagacaguuuugaaaauuuggaagaagaauccaaugaaagugguagcccuuuugacccuguuuuugaaguugaaccuaaaaucucuaacgauaacccagaggaacauguacugaagguaauuccugaggaugcuucagaaucugaggagaagcuagaccaaaaagaggaugguucaaaauacgaaacuauucauuugacugaggaaccaaccaaacuaaugcacaaugcaucugauagugagguugaccaagacgauguuguugaguggaaagacggugcuucuccaucugagagugggccuggaucccaacaagugucagacuuugaggacaauaccugcgaaaugaaaccaggaaccuggucugacgagucuucccaaagcgaagaugcaaggagcaguaagccagcugccaaaaaaaaggcuaccaugcaaggugacagagagcaguugaaauggaagaauaguuccuauggaaaaguugaaggguuuuggucuaaggaccagucacaguggaagaaugcaucugagaaugaugagcgcuuaucuaacccccagauugaguggcagaauagcacaauugacagugaggauggggaacaguuugacaacaugacugauggaguagcugagcccaugcauggcagcuuagccggaguuaaacugagcagccaacaggccuaagugccagguucccuggcauuggugacaugcugcagccuggaacucugaucuccagugugacugcaaagcugucuucucacugguacugccuugugaguacugguuggacuguggggcauguggccgcugcaguuccagugguuauuucuaagucuaugacaggacaggcuguucuugcuucagaaccuucucugacagacacgguaacuaaaugugaaaaaccaauaagcuggugacucaugaauacacacgaggaaaagcagagguuuauuuuaucugccuuuucaacauuucuuucccucugugaaaugauuggucagaugucuuugagaaguguuaaacuaauucacaugguaguguagggccaacauacaagcuaccagucuaauguguauaguagacuuugggaaaagcgauuuuuuuucauguauucauucugaauaguugaaauguauauuuguacagucuuuuagaccuauucaagugaugcucaugauccuguuacugugugcccaucauagauuucuuuuuuuaguguugcccuugcuguguaauaaacgcucuaucuaguuuaccuagcaaaagcucaaaacugcgcuaguauggacuuuuuggacagacuuaguuuuugcacauaaccuuguacaaucuugcaacagaggccagccacguaagauauauaucuggacucucuuguauuauaggauuuuucuuguucugaauauccuugacauuacagcugucaaaaacaaaaacugguauuucagaucuguuuucugaaaucuuuuaagcuaaaaucacaugcaagaauugacuuugcagcuacuaauuuugacaccuuuuagaucuguauaaaaguguguuguguugaagcagcaaaccaaugagugcugcauuuuggauauuuaguuuuaucuuuaguucaacaccaucaugguggauucauuuauaccaucuaauauaugacacacuguuguaguauguauaauuuugugaucuuuauuuucccuuuguauucauuuuaagcaucuaaauaaauugcuguauugugcuuaaugu

SEQ ID NO: 2 - protein, 1102 aa, homo sapiens
MFQLPVNNLGSLRKARKTVKKILSDIGLEYCKEHIEDFKQFEPNDFYLKNTTWEDVGLWDPSLTKNQDYRTKPFCCSACPFSSKFFSAYKSHFRNVHSEDFENRILLNCPYCTFNADKKTLETHIKIFHAPNASAPSSSLSTFKDKNKNDGLKPKQADSVEQAVYYCKKCTYRDPLYEIVRKHIYREHFQHVAAPYIAKAGEKSLNGAVPLGSNAREESSIHCKRCLFMPKSYEALVQHVIEDHERIGYQVTAMIGHTNVVVPRSKPLMLIAPKPQDKKSMGLPPRIGSLASGNVRSLPSQQMVNRLSIPKPNLNSTGVNMMSSVHLQQNNYGVKSVGQGYSVGQSMRLGLGGNAPVSIPQQSQSVKQLLPSGNGRSYGLGSEQRSQAPARYSLQSANASSLSSGQLKSPSLSQSQASRVLGQSSSKPAAAATGPPPGNTSSTQKWKICTICNELFPENVYSVHFEKEHKAEKVPAVANYIMKIHNFTSKCLYCNRYLPTDTLLNHMLIHGLSCPYCRSTFNDVEKMAAHMRMVHIDEEMGPKTDSTLSFDLTLQQGSHTNIHLLVTTYNLRDAPAESVAYHAQNNPPVPPKPQPKVQEKADIPVKSSPQAAVPYKKDVGKTLCPLCFSILKGPISDALAHHLRERHQVIQTVHPVEKKLTYKCIHCLGVYTSNMTASTITLHLVHCRGVGKTQNGQDKTNAPSRLNQSPSLAPVKRTYEQMEFPLLKKRKLDDDSDSPSFFEEKPEEPVVLALDPKGHEDDSYEARKSFLTKYFNKQPYPTRREIEKLAASLWLWKSDIASHFSNKRKKCVRDCEKYKPGVLLGFNMKELNKVKHEMDFDAEWLFENHDEKDSRVNASKTADKKLNLGKEDDSSSDSFENLEEESNESGSPFDPVFEVEPKISNDNPEEHVLKVIPEDASESEEKLDQKEDGSKYETIHLTEEPTKLMHNASDSEVDQDDVVEWKDGASPSESGPGSQQVSDFEDNTCEMKPGTWSDESSQSEDARSSKPAAKKKATMQGDREQLKWKNSSYGKVEGFWSKDQSQWKNASENDERLSNPQIEWQNSTIDSEDGEQFDNMTDGVAEPMHGSLAGVKLSSQQA

SEQ ID NO: 3 - protein, 9 aa, homo sapiens
SALLRSIPA

SEQ ID NO: 4 - protein, 8 aa, homo sapiens
NAPVSIPQ

SEQ ID NO: 5 - protein, 10 aa, homo sapiens
GSALLRSIPA

SEQ ID NO: 6 - protein, 14 aa, homo sapiens
VLGGGSALLRSIPA

SEQ ID NO: 7 - protein, 19 aa, homo sapiens
VEEGIVLGGGSALLRSIPA

SEQ ID NO: 8 - protein, 9 aa, homo sapiens
TALLRTIPA

SEQ ID NO: 9 - protein, 33 aa, homo sapiens
STLSFDLTLQQGSHTNIHLLVTTYNLRDAPAES